(19) United States
(12) Patent Application Publication
(10) Pub. No.: US 2024/0277265 A1
(43) Pub. Date: Aug. 22, 2024

(54) METHOD AND APPARATUS FOR MEASURING NON-CONTACT MULTIPLE BIOSIGNAL

(71) Applicant: ELECTRONICS AND TELECOMMUNICATIONS RESEARCH INSTITUTE, Daejeon (KR)

(72) Inventors: Chul HUH, Daejeon (KR); Jeong Won PARK, Seoul (KR); Hong Seok SEO, Daejeon (KR)

(21) Appl. No.: 18/419,785

(22) Filed: Jan. 23, 2024

(30) Foreign Application Priority Data
Feb. 22, 2023 (KR) 10-2023-0023392

(51) Int. Cl. A61B 5/1455; A61B 5/00; A61B 5/024

(52) U.S. Cl. CPC A61B 5/14552; A61B 5/0082; A61B 5/02427; A61B 5/7235

(57) ABSTRACT

A method and an apparatus for measuring a non-contact multiple biosignal are provided. An apparatus for measuring a non-contact multiple biosignal includes: one or more processors; and memory storing instructions that, when executed by the one or more processors, cause the apparatus to: radiate first emission light of a first wavelength and second emission light of a second wavelength toward an object to be measured; receive first reflected light of the first wavelength and second reflected light of the second wavelength reflected from the object to be measured; detect first light absorbance for the first reflected light and second light absorbance for the second reflected light; and determine oxygen saturation of the object to be measured based on a difference between the first light absorbance and the second light absorbance.

### CROSS-REFERENCE TO RELATED APPLICATION

This application claims the benefit of priority to Korean Patent Application No. 10-2023-0023392, filed in the Korean Intellectual Property Office on Feb. 22, 2023, the entire contents of which are incorporated herein by reference.

### BACKGROUND

### 1. Technical Field

The present disclosure relates to a method and an apparatus for measuring a non-contact multiple biosignal.

### 2. Description of Related Art

The description in this section merely provides background information related to the present disclosure and does not necessarily constitute the related art.

When an infectious disease such as COVID-19 occurs, it is very important to screen people with symptoms of the infectious disease quickly. This is because when the confirmation of an infectious disease such as COVID-19 is delayed, the risk of contagion to others greatly increases.

Currently, an infectious disease such as COVID-19 is confirmed by collecting samples from the nasal cavity using molecular diagnostic technology and then testing for the coronavirus. However, in the case of molecular diagnostic tests, it requires a long time of about 4 hours or more because it goes through processes such as sample collection, pre-processing, and amplification, and also has drawbacks such as pain and discomfort for a subject when collecting samples and a long wait to find out whether an infectious disease is confirmed.

Infection with COVID-19 virus is accompanied by a variety of complex physical symptoms such as fever, cough, difficulty breathing, and sore throat. Among these various symptoms, the only symptom currently used to screen people with symptoms infected with the COVID-19 virus is measuring body temperature.

However, even when infected by the COVID-19 virus, only about 25% of all infected people show fever symptoms. Accordingly, by simultaneously measuring multiple biological signals (body temperature, oxygen saturation, pulse, etc.) related to various complex physical symptoms that appear when infected with the COVID-19 virus, people with symptoms infected with the COVID-19 virus may be screened early with very high accuracy. Accordingly, it is very effective in preventing the large-scale spread of the COVID-19 virus.

Among various biosignals, oxygen saturation (SpO2) refers to the amount of oxygen combined with hemoglobin (Hb) in red blood cells. Using oxygen saturation in the blood, it is possible to determine effective breathing or whether oxygen is being delivered well to the human body through the amount of oxygen carried by red blood cells. Oxygen saturation is expressed as a percentage of the concentration ratio of oxidized hemoglobin combined with oxygen to the total hemoglobin concentration. The normal level of oxygen saturation is 95% or higher. When the oxygen saturation falls below this level, it may cause hypoxia, which may lead to an emergency situation where breathing becomes difficult.

The most accurate method to measure oxygen saturation is to collect arterial blood and measure blood oxygen saturation. However, this method requires blood collection and has the drawback of requiring a certain amount of time to analyze the oxygen saturation value after blood collection.

The most representative non-invasive method that does not require blood collection is pulse oximetry, which measures oxygen saturation through absorbance using an optical device.

The currently used pulse oximetry method uses a technology to measure oxygen saturation through contact by attaching a device to the tip of a finger.

However, this contact-based measurement method is inappropriate for screening people with symptoms of highly contagious infectious diseases such as the COVID-19 virus. This is because the contact method is impossible to prevent the spread of viruses through used devices because many people use the same device, and additional processes such as device sterilization are required to prevent virus spread. Accordingly, it is necessary to develop non-contact multiple biosignal measurement technology to efficiently screen people with symptoms infected with the COVID-19 virus early and to monitor confirmed patients at all times.

Currently, technology for measuring pulse and oxygen saturation from the body in a non-contact manner is being developed using various methods such as light, electromagnetic waves, and images. However, it is in the basic technology development stage and has the drawback of low measurement accuracy.

### SUMMARY

The following summary presents a simplified summary of certain features. The summary is not an extensive overview and is not intended to identify key or critical elements.

An aspect of the present disclosure is to provide a method and an apparatus for measuring a non-contact multiple biosignal.

The aspects of the present disclosure are not limited to those mentioned above, and other aspects not mentioned herein will be clearly understood by those skilled in the art from the following description.

An apparatus may comprise: one or more processors; and memory storing instructions that, when executed by the one or more processors, cause the apparatus to: radiate first emission light of a first wavelength and second emission light of a second wavelength toward an object to be measured; receive first reflected light of the first wavelength and second reflected light of the second wavelength reflected from the object to be measured; detect first light absorbance for the first reflected light and second light absorbance for the second reflected light; and determine oxygen saturation of the object to be measured based on a difference between the first light absorbance and the second light absorbance.

A method, may comprise: radiating first emission light of a first wavelength and second emission light of a second wavelength toward an object to be measured; receiving first reflected light of the first wavelength and second reflected light of the second wavelength reflected from the object to be measured; detecting first light absorbance for the first reflected light and second light absorbance for the second reflected light; and determining oxygen saturation of the object to be measured according to a difference between the first light absorbance and the second light absorbance.

An embodiment of the present disclosure is directed to a sensor device capable of measuring bio-signals reflected from nails of the human body in a non-contact manner using an optical device and a light detector, and has the benefit of being simple to manufacture.

In addition, according to an embodiment of the present disclosure, a biosignal measurement sensor using the optical device and the light detector does not require a complicated optical system and may be used to quantitatively detect pulse and oxygen saturation simultaneously with high sensitivity.

The benefits of the present disclosure are not limited to those mentioned above, and other benefits not mentioned herein will be clearly understood by those skilled in the art from the following description.

These and other features and advantages are described in greater detail below.

### BRIEF DESCRIPTION OF THE DRAWINGS

FIG. 1 is an exemplary functional block diagram illustrating an apparatus for measuring a biosignal.

FIG. 2 is a diagram illustrating a first absorption spectrum showing a change in light absorption of a first wavelength over time with respect to first reflected light.

FIG. 3 is a graph showing the principle of measuring oxygen saturation from the first absorption spectrum and a second absorption spectrum.

FIG. 4 is a flowchart illustrating a method for measuring a biosignal using the apparatus for measuring the biosignal.

### DETAILED DESCRIPTION

Hereinafter, some examples of the present disclosure will be described in detail with reference to the accompanying drawings. In the following description, like reference numerals preferably designate like elements, although the elements are shown in different drawings. Further, in the following description of some examples, a detailed description of related known components and functions when considered obscuring the subject of the present disclosure will be omitted for the purpose of clarity and for brevity.

Additionally, various terms such as first, second, A, B, (a), (b), etc., are used solely for the purpose of differentiating one component from others but not to imply or suggest the substances, the order or sequence of the components. Throughout this specification, when parts “include” or “comprise” a component, they are meant to further include other components, not excluding thereof unless there is a particular description contrary thereto. The terms such as “unit,” “module,” and the like refer to units for processing at least one function or operation, which may be implemented by hardware, software, or a combination thereof.

The detailed description set forth below in conjunction with the appended drawings is intended to describe exemplary examples of the present disclosure and is not intended to represent the only examples in which the present disclosure may be practiced.

As used below, singular terms may include plural terms unless otherwise specified.

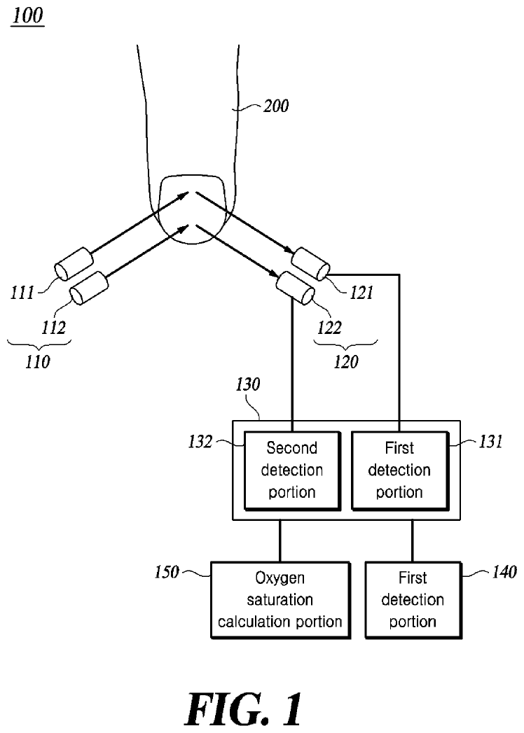

FIG. 1 is an exemplary functional block diagram illustrating an apparatus for measuring a biosignal according to an embodiment.

An apparatus 100 for measuring a biosignal 100 according to an embodiment includes a light source irradiation portion 110, a reflected light reception portion 120, a light absorption detection portion 130, a pulse rate calculation portion 140, and an oxygen saturation calculation portion 150.

The light source irradiation portion 110 radiates first emission light of a first wavelength and second emission light of a second wavelength toward an object 200 to be measured.

Herein, the object 200 to be measured may be a fingernail of the human body.

The light source irradiation unit 110 includes a first light source 111 and a second light source 112. The first light source 111 and the second light source 112 may each be used as light emitting diodes that generate emission light of different wavelengths.

The first light source 111 generates first emission light with a wavelength of 660 nm, which is the first wavelength, and the second light source 112 generates second emission light with a wavelength of 940 nm, which is the second wavelength.

The reflected light reception portion 120 receives the first reflected light of the first wavelength and the second reflected light of the second wavelength reflected from the object 200 to be measured.

The reflected light reception portion 120 includes a first light reception portion 121 and a second light reception portion 122. A Complementary Metal-Oxide Semiconductor (CMOS) image sensor may be used as the first light reception portion 121 and the second light reception portion 122, respectively.

In order to be in a non-contact state with the object 200 to be measured of the human body, which is the object for measuring pulse rate and oxygen saturation, the positions of the first light source 111 and the second light source 112, and the first light reception portion 121 and the second light reception portion 122 are configured to be a certain distance away from the position of the object 200 to be measured.

Herein, the first light reception portion 121 may be formed as a CMOS image sensor for absorbing reflected light of the first wavelength, and the second light reception portion 122 may be formed as a CMOS image sensor for absorbing reflected light of the second wavelength.

The light absorption detection portion 130 detects the first light absorbance for the first reflected light and the second light absorbance for the second reflected light.

The light absorption detection portion 130 may include a first detection portion 131 and a second detection portion 132.

The first detection portion 131 detects the light absorbance for the first wavelength for the first reflected light received from the first light reception portion 121 for a certain period of time and stores the same as a first absorption spectrum, and the second detection portion 132 detects the light absorption for the second wavelength for the second reflected light received from the second light reception portion 122 for a certain period of time and stores the same as a second absorption spectrum.

Among the light incident on the area of the object 200 to be measured from the first light source 111 and the second light source 112, which are two light sources with different wavelengths, a certain portion of the light is absorbed by the microvessels inside the object 200 to be measured, the remaining light that is not absorbed is reflected, and a portion of the reflected light is incident on the first light reception portion 121 and the second light reception portion 122.

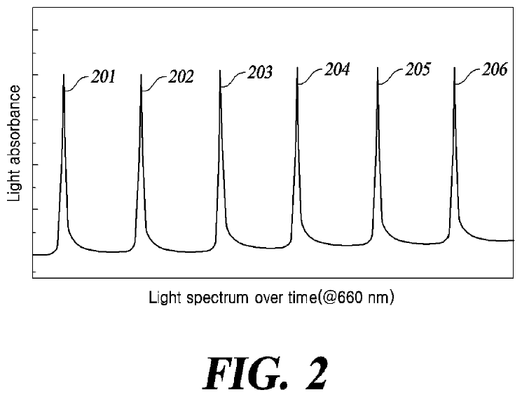

FIG. 2 is a diagram illustrating a first absorption spectrum showing a change in light absorption of a first wavelength over time with respect to first reflected light.

The graph of FIG. 2 shows that the first detection portion 131 detects the light absorbance peak values 201, 202, 203, 204, and 205 for the first wavelength for the first reflected light incident on the first light reception portion 121 for a certain period of time and stores the same as the first absorption spectrum.

Blood flow within the microvessels inside the object 200 to be measured changes with time, and this change in blood flow is related to the pulse rate of the object 200 to be measured. In other words, when the pulse rate of the object 200 to be measured is fast, the change in blood flow within the microvessels is fast, and when the pulse rate of the object 200 to be measured is slow, the change in blood flow is slow.

Referring to FIG. 2, when light is incident on the object 200 to be measured from the first light source 111 that emits light with a wavelength of 660 nm, which is the first wavelength, the light absorbance spectrum of light incident on the first light reception portion 121 changes depending on the change in blood flow within the microvessels inside the object 200 to be measured.

Accordingly, the pulse rate of the object 200 to be measured may be calculated by analyzing the first absorption spectrum of the reflected light incident on the first light reception portion 121.

The pulse rate calculation portion 140 calculates the pulse rate of the object 200 to be measured from the time interval of the change in a first light absorbance peak value from the first absorption spectrum.

For example, in FIG. 2, the pulse rate calculation portion 140 checks the number of occurrences of the light absorbance peak values 201, 202, 203, 204, 205, and 206 and the time required corresponding to the number of occurrences, and calculates the pulse rate of the object 200 to be measured by dividing the number of occurrences of the first light absorbance peak value by the corresponding time required. For example, assuming that the time between the first light absorbance peak value 201 and the sixth light absorbance peak value 206 is 10 seconds, the light absorbance peak value occurred 5 times during 10 seconds. Accordingly, the pulse rate calculation portion 140 calculates that the pulse rate per minute is 30 times using Equation 2 as follows.

$$\\ \begin{matrix}Pulse rate = \frac{C}{T} & [ Equation 1 ]\end{matrix}$$

Herein, C is the number of occurrences of the light absorption peak value, and T is the time required.

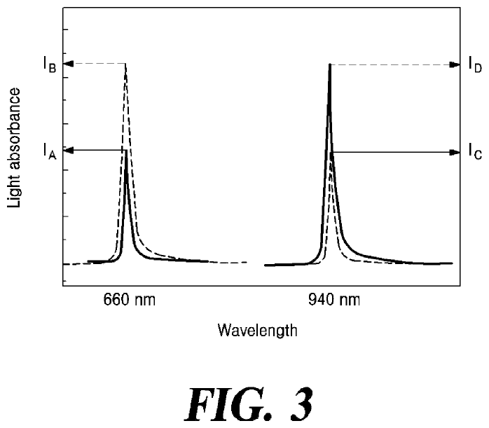

FIG. 3 is a graph showing the principle of measuring oxygen saturation from the first absorption spectrum and a second absorption spectrum.

The second detection portion 132 detects the light absorbance for the second wavelength for the second reflected light received from the second light reception portion 122 for a certain period of time and stores the same as a second absorption spectrum. The graph form of the second absorption spectrum is similar to that of the first absorption spectrum. However, as illustrated in FIG. 3, the size of each light absorption peak value in the first absorption spectrum and the second absorption spectrum is different in form in the case of hemoglobin with which oxygen is combined within the object 200 to be measured and hemoglobin with which oxygen is not combined.

The oxygen saturation calculation portion 150 calculates the oxygen saturation of the object 200 to be measured according to the difference between the first light absorbance at the first wavelength and the second light absorbance at the second wavelength.

After the first and second emission lights are incident on the object 200 to be measured from the first light source 111 and the second light source 112, which are two optical devices having each different wavelength (660 nm and 940 nm), according to the change in the peak value of light absorbance for the reflected light of two different wavelengths (660 nm, 940 nm) reflected from the object 200 to be measured, the oxygen saturation calculation portion 150 may calculate the oxygen saturation inside the microvessels below the surface of the object 200 to be measured.

The light absorbance peak values of hemoglobin with which oxygen is combined and hemoglobin with which oxygen is not combined are different from each other in reflected light of different wavelengths.

The oxygen saturation calculation portion 150 calculates the oxygen saturation of the object 200 to be measured by detecting the light absorbance peak values (IA, IB, IC, ID) of hemoglobin (HbO2) combined with oxygen and hemoglobin (Hb) not combined with oxygen from the first absorption spectrum and the second absorption spectrum, respectively.

In other words, in the 660 nm wavelength range, the absorbance (IB) of hemoglobin combined with oxygen is much larger than the light absorbance peak value (IA) of hemoglobin not combined with oxygen, and in the 940 nm wavelength range, the light absorbance peak value (ID) of hemoglobin not combined with oxygen is much larger than the light absorbance peak value (IC) of hemoglobin combined with oxygen. Accordingly, it is possible to accurately and quantitatively calculate the oxygen saturation within the microvessels inside the surface of the object 200 to be measured using the change in light absorption peak values (IA, IB, IC, ID) at different wavelengths (660 nm, 940 nm).

As illustrated in FIG. 3, the oxygen saturation calculation portion 150 may calculate the oxygen saturation by analyzing the relative intensity change of the first reflected light and the second reflected light having wavelengths of 660 nm and 940 nm, respectively.

In order to analyze the relative intensity change of the two reflected lights, the oxygen saturation calculation portion 150 calculates oxygen saturation by calculating a first statistical index of the first light absorbance peak value in the first spectrum and a second statistical index of the second light absorbance peak value in the second spectrum.

Herein, the standard deviation for a plurality of first light absorbance peak values in the first spectrum divided by the average of the first light absorbance peak values is determined as the first statistical index. In addition, the standard deviation for a plurality of second light absorbance peak values in the second spectrum divided by the average of the second light absorbance peak values is determined as the second 5 statistical index.

The oxygen saturation calculation portion 150 calculates the oxygen saturation based on the ratio between the first statistical index and the second statistical index.

In other words, the oxygen saturation calculation portion 150 may calculate the oxygen saturation SpO₂ according to Equation 2 below.

$$\\ \begin{matrix}Sp {O}_{2} = {K}_{1} - {K}_{2} \times ( \frac{{a}_{1}}{{a}_{2}} ) & [ Equation 2 ]\end{matrix}$$

Herein, K₁ and K₂ are proportionality constants and may be set to 110 and 25, respectively, without being limited thereto.

In addition, a₁ is a first statistical index and represents the standard deviation for a plurality of first light absorbance peak values divided by the average of the first light absorbance peak values, and a₂ is a second statistical index and represents the standard deviation for a plurality of second light absorbance peak values divided by the average of the first light absorbance peak values.

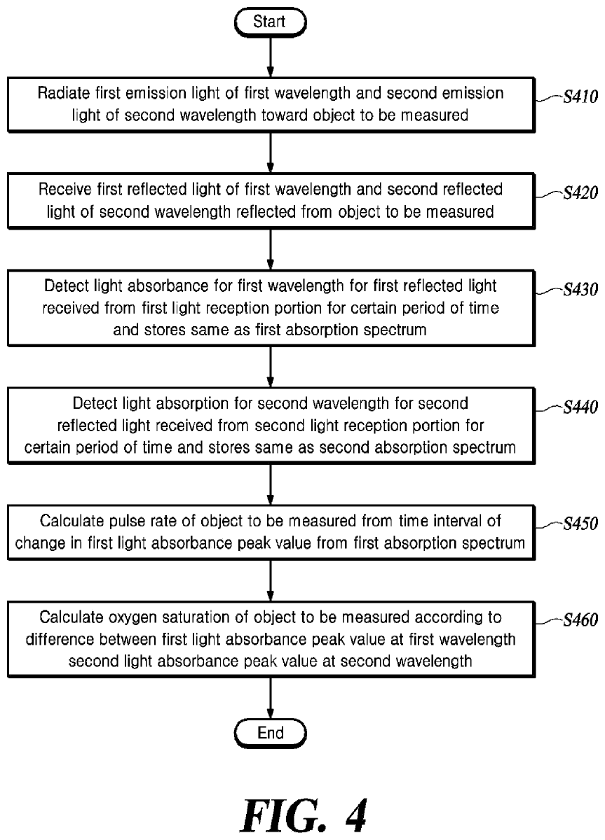

FIG. 4 is a flowchart illustrating a method for measuring a biosignal using the apparatus for measuring the biosignal.

Hereinafter, a method for measuring a biosignal will be described with reference to FIGS. 1 to 4.

The light source irradiation portion 110 radiates first emission light of the first wavelength and second emission light of the second wavelength toward the object 200 to be measured (S410).

The reflected light reception portion 120 receives the first reflected light of the first wavelength and the second reflected light of the second wavelength reflected from the object 200 to be measured (S420).

The first detection portion 131 detects the light absorbance for the first wavelength for the first reflected light received to the first light reception portion 121 for a certain period of time and stores the same as a first absorption spectrum (S430).

The second detection portion 132 detects the light absorption for the second wavelength for the second reflected light received to the second light reception portion 122 for a certain period of time and stores the same as a second absorption spectrum (S440).

The pulse rate calculation portion 140 calculates the pulse rate of the object 200 to be measured from the time interval of the change in a first light absorbance peak value from the first absorption spectrum (S450).

The oxygen saturation calculation portion 150 calculates the oxygen saturation of the object 200 to be measured according to the difference between the first light absorbance peak value at the first wavelength and the second light absorbance peak value at the second wavelength (S460).

In order to analyze the intensity change of the relative peak value of two reflected lights, the oxygen saturation calculation portion 150 determines the standard deviation of the first light absorbance peak value in the first spectrum divided by the average of the first light absorbance peak value as the first statistical index.

In addition, the oxygen saturation calculation portion 150 determines the standard deviation of the second light absorbance peak value in the second spectrum divided by the average of the second light absorbance peak value as the second statistical index.

The oxygen saturation calculation portion 150 calculates the oxygen saturation based on the value obtained by dividing the first statistical index by the second statistical index as shown in Equation 2 above.

Since the operations of the pulse rate calculation portion 140 and the oxygen saturation calculation portion 150 have been described above, further description thereof will be omitted.

According to one or more aspects of the disclosure, an apparatus for measuring a biosignal may comprise: a light source irradiation portion that radiates first emission light of a first wavelength and second emission light of a second wavelength toward an object to be measured; a reflected light reception portion that receives first reflected light of the first wavelength and second reflected light of the second wavelength reflected from the object to be measured; a light absorbance detection portion that detects first light absorbance for the first reflected light and second light absorbance for the second reflected light; and an oxygen saturation calculation portion that calculates oxygen saturation of the object to be measured according to a difference between the first light absorbance and the second light absorbance.

The apparatus may further comprise a pulse rate calculation portion that calculates a pulse rate of the object to be measured from a time interval of a change in the first light absorbance.

The light absorbance detection portion may detect the first light absorbance and the second light absorbance over time.

The oxygen saturation calculation portion may calculate the oxygen saturation using a first statistical index of the first light absorbance and a second statistical index of the second light absorbance.

The oxygen saturation calculation portion may calculate the oxygen saturation based on a ratio between the first statistical index and the second statistical index.

A method for measuring a biosignal may comprise one or more operations described above. The method may be implemented in a non-transitory computer-readable medium storing instructions that, when executed, cause performance of one or more operations described herein.

The components described in the example embodiments may be implemented by hardware components including, for example, at least one digital signal processor (DSP), a processor, a controller, an application-specific integrated circuit (ASIC), a programmable logic element, such as an FPGA, other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers. A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors.

Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment. Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

What is claimed is:

1. An apparatus comprising:
one or more processors; and
memory storing instructions that, when executed by the one or more processors, cause the apparatus to:
radiate first emission light of a first wavelength and second emission light of a second wavelength toward an object to be measured;
receive first reflected light of the first wavelength and second reflected light of the second wavelength reflected from the object to be measured;
detect first light absorbance for the first reflected light and second light absorbance for the second reflected light; and
determine oxygen saturation of the object to be measured based on a difference between the first light absorbance and the second light absorbance.

2. The apparatus of claim 1, wherein the instructions, when executed by the one or more processors, cause the apparatus to determine a pulse rate of the object to be measured from a time interval of a change in the first light absorbance.

3. The apparatus of claim 1, wherein the instructions, when executed by the one or more processors, cause the apparatus to detect the first light absorbance and the second light absorbance over time.

4. The apparatus of claim 3, wherein the instructions, when executed by the one or more processors, cause the apparatus to determine the oxygen saturation using a first statistical index of the first light absorbance and a second statistical index of the second light absorbance.

5. The apparatus of claim 4, wherein the instructions, when executed by the one or more processors, cause the apparatus to determine the oxygen saturation based on a ratio between the first statistical index and the second statistical index.

6. The apparatus of claim 5, wherein:
the first statistical index is a first value obtained by dividing a standard deviation of a plurality of the first light absorbances by the average of the first light absorbance; and
the second statistical index is a second value obtained by dividing a standard deviation of a plurality of the second light absorbances by the average of the second light absorbance.

7. The apparatus of claim 6, wherein the oxygen saturation is determined by an equation below:
$$\\ {K}_{1} - {K}_{2} * [ {a}_{1} / {a}_{2} ] ,$$
where K₁ and K₂ are constants, a₁ is the first value, and a₂ is the second value.

8. The apparatus of claim 1, wherein the first wavelength is 660 nm and the second wavelength is 940 nm.

9. A method comprising:
radiating first emission light of a first wavelength and second emission light of a second wavelength toward an object to be measured;
receiving first reflected light of the first wavelength and second reflected light of the second wavelength reflected from the object to be measured;
detecting first light absorbance for the first reflected light and second light absorbance for the second reflected light; and
determining oxygen saturation of the object to be measured according to a difference between the first light absorbance and the second light absorbance.

10. The method of claim 9, further comprising determining a pulse rate of the object to be measured from a time interval of a change in the first light absorbance.

11. The method of claim 9, wherein the first light absorbance and the second light absorbance are each detected over time.

12. The method of claim 11, wherein the oxygen saturation is determined using a first statistical index of the first light absorbance and a second statistical index of the second light absorbance.

13. The method of claim 12, wherein the oxygen saturation is determined based on a ratio between the first statistical index and the second statistical index.

14. The method of claim 13, wherein:
the first statistical index is a first value obtained by dividing a standard deviation of a plurality of the first light absorbances by the average of the first light absorbance; and
the second statistical index is a second value obtained by dividing a standard deviation of a plurality of the second light absorbances by the average of the second light absorbance.

15. The method of claim 14, wherein the oxygen saturation is determined by an equation below:
$$\\ {K}_{1} - {K}_{2} * [ {a}_{1} / {a}_{2} ] ,$$
where K₁ and K₂ are constants, a₁ is the first value, and a₂ is the second value.

16. The method of claim 15, wherein the K₁ is 110 and the K₂ is 25.
